(19) United States
(12) Patent Application Publication
(10) Pub. No.: US 2008/0139615 A1
(43) Pub. Date: Jun. 12, 2008

(54) COMBINATION OF A HYPNOTIC AGENT AND R (+)-ALPHA-(2,3-DIMETHOXY-PHENYL)-1-[2-(4-FLUOROPHENYL)ETHYL]-4-PIPERIDINEMETHANOL AND THERAPEUTIC APPLICATION THEREOF

(75) Inventors: Gary T. EMMONS, Washington, NJ (US); Sathapana KONGSAMUT, Madison, NJ (US); Craig N. KARSON, Wayne, PA (US); Corinne M. LEGOFF, Montclair, NJ (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMACEUTICALS INC., Bridgewater, NJ (US)

(21) Appl. No.: 12/027,012

(22) Filed: Feb. 6, 2008

Related U.S. Application Data
(63) Continuation of application No. PCT/US2006/032026, filed on Aug. 16, 2006.
(60) Provisional application No. 60/709,510, filed on Aug. 19, 2005.

(51) Int. Cl. A61K 31/445; A61K 31/425; A61K 31/405; A61K 31/5513

(52) U.S. Cl. 514/317; 514/419; 514/373; 514/221

(57) ABSTRACT

The invention concerns the combination of a short-acting hypnotic agent and R-(+)-α-(2,3-dimethoxyphenyl)-1-[2-(4-fluorophenyl)ethyl]-4-piperidinemethanol (Compound A) or its prodrug having the Formula II:

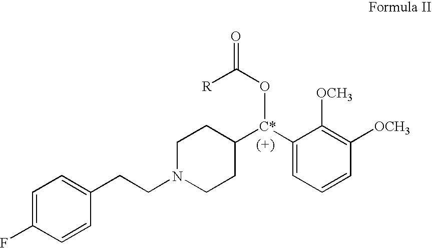

wherein R is C₁-C₂₀ alkyl or a pharmaceutically acceptable salt thereof. The combination of this invention is useful in treating a variety of sleep disorders.

### CROSS REFERENCE TO RELATED APPLICATIONS

This application is a continuation of International application No. PCT/US2006/032,026, filed Aug. 16, 2006, which is incorporated herein by reference in its entirety; which claims the benefit of priority of U.S. Provisional Application No. 60/709,510, filed Aug. 19, 2005.

### BACKGROUND OF THE INVENTION

1. Field of the Invention

The present invention relates to a combination of at least one hypnotic agent with R (+)-α-(2,3-dimethoxy-phenyl)-1-[2-(4-fluorophenyl)ethyl]-4-piperidinemethanol. The combination of this invention is useful in the treatment of a variety of sleep disorders.

2. Description of the Art

Chronic insomnia among adults in the United States has been estimated to be present in ten percent of the adult population, and the annual cost for its treatment is estimated at $10.9 billion. JAMA 1997; 278: 2170-2177 at 2170. Chronic insomniacs report elevated levels of stress, anxiety, depression and medical illnesses. The most common class of medications for treating insomnia are the benzodiazepines, but the adverse effect profile of benzodiazepines include daytime sedation, diminished motor coordination, and cognitive impairments. Furthermore, the National Institutes of Health Consensus conference on Sleeping Pills and Insomnia in 1984 have developed guidelines discouraging the use of such sedative-hypnotics beyond 4-6 weeks because of concerns raised over drug misuse, dependency, withdrawal and rebound insomnia. JAMA 1997; 278: 2170-2177 at 2170. Therefore, it is desirable to have a pharmacological agent for the treatment of insomnia which is more effective and/or has fewer side effects than those currently used.

The prevalence of obstructive sleep apnea is estimated to be approximately 1-10% in the adult population, but may be higher in elderly individuals; Diagnostic and Statistical Manual of Mental Disorders 4^{th} ed., American Psychiatric Association, Washington D.C. (1994). Preliminary evidence suggests that having obstructive sleep apnea may contribute to increased susceptibility to cardiovascular complications such as hypertension, cardiac arrhythmias, stroke, and myocardial infarction. Excessive daytime sleepiness is also a major complication.

Currently, the therapies used to treat obstructive sleep apnea include weight loss for the obese patient, Nasal-continuous positive Airway Pressure (a facemask used at night which produces a positive pressure within the upper airway), pharyngeal surgery and the administration of a variety of pharmacologic agents which have not been proven to be entirely successful. Chest 109 (5): 1346-1358 (May 1996) entitled “Treatment of Obstructive Sleep Apnea”, a Review, hereby incorporated by reference. These agents include acetazolamide, medroxyprogesterone, opioid antagonists, nicotine, angiotensin-converting enzyme inhibitors and psychotropic agents (including those that prevent the reuptake of biogenic amines such as norepinephrine, dopamine and serotonin). Id. at 1353. Many of these pharmacological agents used also have a ventilatory depressant action (such as benzodiazepines) or other side effects such as urinary hesitancy and/or impotence in men (protriptyline) so that a new agent with fewer side effects is needed for the treatment of obstructive sleep apnea. Even though serotonin is a sleep-inducing agent and may be a ventilatory stimulant (Id. at 1354), 5HT_{2A} receptor antagonists have been found useful in treating obstructive sleep apnea. See also Am. J. Respir Crit. Care Med (153) pp 776-786 (1996) where serotonin antagonists exacerbated sleep apnea produced in English bulldogs. But compare, Journal of Physiology (466) pp 367-382 (1993), where it is postulated that an excess of serotonin due to dysfunction of the serotonin biosynthesis mechanisms might set up conditions which favor obstructive apneas; European Journal of Pharmacology (259):71-74 (1994) further work on rat model with 5HT₂ antagonist.

EP 1 262 197 discloses a method of treating sleep disorders including sleep apnea by administering to a patient in need of such a treatment a 5HT_{1A} antagonist or an alpha-2-adrenergic antagonist in combination with an antidepressant such as serotonin reuptake inhibitor (SRI). Such a combination exhibits an improvement in efficacy.

U.S. Pat. No. 6,143,792 discloses that a specific 5HT_{2A} receptor antagonist is useful in the treatment of the sleep apnea syndrome. Similarly, U.S. Pat. No. 6,576,670 discloses that a specific 5HT_{2A} and 5HT_{2A/C} receptor antagonist is useful in the treatment of snoring and upper airway high resistance syndrome.

The compound R-(+)-α-(2,3-dimethoxyphenyl)-1-[2-(4-fluorophenyl)ethyl]-4-piperidinemethanol (hereafter referred to as the “Compound A”) is a 5HT_{2A} antagonist useful in the treatment of a variety of disorders. U.S. Pat. No. 5,169,096 claimed compounds having a generic scope which encompassed the Compound A and disclosed uses of the treatment of anorexia nervosa, variant angina, Raynaud's phenomenon, coronary vasospasms, prophylactic treatment of migraine, cardiovascular diseases such as hypertension, peripheral vascular disease, thrombotic episodes, cardiopulmonary emergencies and arrhythmias, and has anesthetic properties. See also U.S. Pat. Nos. 4,783,471; 4,912,117; and 5,021,428, which are divisions of U.S. Pat. No. 5,169,096. See also U.S. Pat. Nos. 4,877,798 (fibromyalgia), 4,908,369 (insomnia); 5,106,855 (glaucoma); EP 319 962 (anxiety); EP 337 136 (extrapyramidal symptoms). All of the foregoing references are incorporated herein by reference.

The Compound A was then specifically claimed in U.S. Pat. No. 5,134,149 which disclosed uses of antagonizing serotonin at the 5HT2 receptor, treating anxiety, variant angina, anorexia nervosa, Raynaud's phenomenon, intermittent claudication, coronary or peripheral vasospasms, fibromyalgia, extrapyramidal symptoms, arrhythmias, thrombotic illness, transient ischemic attacks, drug abuse, and psychotic illness such as schizophrenia and mania. See also U.S. Pat. Nos. 5,561,144; 5,700,812; 5,700,813; 5,721,249—divisionals of U.S. Pat. No. 5,134,149- and also U.S. Pat. No. 5,618,824 (obsessive compulsive disorder) and U.S. Pat. No. 6,022,877 and (depressive disorders including major depressive episode and dysthymia, and bipolar disorder).

The Compound A is highly selective in its activity at the 5HT2A receptor compared to other receptors, and, as such, has reportedly fewer side effects. It has been shown to have a better CNS safety index relative to the reference compounds haloperidol, clozapine, risperidone, ritanserin, and amperozide in preclinical testing. JPET 277:968-981, 1996, incorporated herein by reference. It has recently been discovered that Compound A is useful in the treatment of sleep disorders such as insomnia and obstructive sleep apnea. See U.S. Pat. Nos. 6,277,864 and 6,613,779. A prodrug of the Compound A has also been disclosed recently. See U.S. Pat. Nos. 6,028,083 and 6,063,793. Recently, a biodegradable polymer encapsulated pharmaceutical composition containing the Compound A has also been disclosed, see U.S. Pat. No. 6,455,526.

A certain number of hypnotic agents, having various modes and acting duration, have also been developed over the years. For instance, a class of hypnotic agents have been developed which are long acting ones. Also, a class of short-acting hypnotic agents has also been developed. Generally, a short acting hypnotic agent acts mainly as a sleep inducer, i.e., the entry time into the sleep phase.

An example of a short acting hypnotic agent include without any limitation, zolpidem, which acts as a modulator of the GABA-A receptors. Zolpidem belongs to the imidazopyridine class and is administered orally in the form of an immediate-release tablet or in a galenic form allowing a delayed release. Zolpidem acts quickly, and is well absorbed with a 70% bioavailability. The average dosage, between 5 and 10 mg in a conventional formulation, induces a maximum plasma concentration which is reached between 0.5 and 3 hours of administration, the half life is short, with an average value of about 2.4 hours and an acting time of up to 6 hours.

Other examples of a short-acting hypnotic agent include without any limitation zaleplon, which belongs to the pyrazolopyrimidine class, zopiclone, eszopiclone, which belong to the cyclopyrrolone class, as well as their derivatives. Various other short acting hypnotic agents have also been developed including phenothiazines and benzodiazepines. Specific compounds belonging to these therapeutic classes include for example triazolam, brotizolam or alimemazine.

Long-acting hypnotic agents and/or sleep aids have also been developed. In the following it is understood that a long-acting hypnotic agent is referred to a compound or agent that is mainly a sleep inducer but may also be capable of improving sleep quality and/or maintenance in a patient. The “sleep aid” is a compound or agent that is mainly used to improve sleep quality and/or sleep maintenance in a patient, in particular the deep sleep phases. One such example of a sleep aid is an inhibitor of the 5HT2A receptors that acts without blockage of the dopamine, such as the Compound A or its prodrug.

Other long-acting hypnotic agents are, for example, temazepam, clonazepam, gaboxadol and pregabaline, a modulator of calcium ion, as well as their derivatives.

The hypnotic agents and/or the sleep aids described above improve sleep disorders, in particular, insomnia. However, whereas the short-acting hypnotic agents act mainly on the sleep-entry phase, the long-acting hypnotic agents act mainly on the sleep-entry phase but may also have a sleep maintenance component and sleep aids act rather on the deep-sleep phase, thus help to improve the overall quality of sleep in a patient.

Particularly, short acting GABAergic agonists such as zopiclone and eszopiclone provide benefits on sleep onset and sleep maintenance. However, optimal sleep maintenance effects may only be seen at doses that create a risk for next-day dysfunction, and which may raise unnecessary risks of memory and gait impairment, and of respiratory dysfunction. Therefore, an agent such as inhibitors of 5HT2A receptors that provides additional sleep maintenance effects, operating through a complementary mechanism, would be desired.

In addition, while zopiclone/eszopiclone do not have the negative effects on stage 3/4 sleep (Slow Wave Sleep; SWS) seen with benzodiazepines, they do not appear to significantly enhance SWS. These stages have been associated with the restorative activity of sleep, and hence enhancement of these stages, which are reduced in patients with sleep maintenance insomnia (at least as compared with young healthy volunteers), may provide improvement in daytime function, and possibly in addressing other disorders associated with aging and sleep deprivation (including increased adiposity, decreased lean body mass, and increased risk for diabetes mellitus) (Van Cauter et al., JAMA, 2000; 284:861-868).

The mechanism of serotonin 2A antagonism (5HT2A) may also facilitate circadian entrainment, an issue in older subjects who tend to have phase advancement and (especially in demented populations) a general disruption of rhythmicity of circadian processes.

It should also be noted that slow wave sleep (SWS) is associated with reduced risk of arousals and awakenings (Salzarulo et al., Sleep Research Online, 1999; 2:73-77). This may be particularly true in older subjects (Boselli. et al., Sleep, 1998; 21:361-367). In addition, in older adult patients with insomnia, diminished SWS has been associated with cognitive impairments (Crenshaw & Edinger, Physiol. Behav., 1999; 66:485-492). Compound A has been established to increase SWS and decrease arousals and sleep stage shifts to wakefulness in patients with sleep maintenance insomnia.

Accordingly, it is an object of this invention to provide a combination, which allows combining the actions of the sleep aids and/or the long and short-acting hypnotic agents by improving the sleep quality and the respective effects of the short and long-acting hypnotic agents and/or sleep aids, without negative effect on the patient's waking-up phases.

Other objects and further scope of the applicability of the present invention will become apparent from the detailed description that follows.

### SUMMARY OF THE INVENTION

Thus in accordance with this invention there is provided a combination of one or more hypnotic agents and one or more sleep aids. The combination of the invention comprises at least a short-acting hypnotic agent and/or a long-acting hypnotic agent and a sleep aid. In accordance with this aspect of the invention, the short and long-acting hypnotic agents are present in a galenic formulation adapted to an immediate or delayed release, and the sleep aid is present in the form of a galenic formulation adapted to an immediate-release.

More particularly, the present invention provides a combination of at least one short acting hypnotic agent with Compound A or its prodrug or a pharmaceutically acceptable salt thereof, wherein the prodrug is of the Formula II;

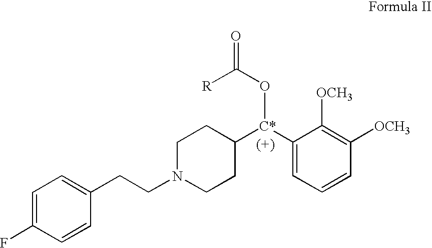

wherein R is C₁-C₂₀ alkyl.

The combination of a short and long-acting hypnotic agents with a sleep aid allows to obtain beneficial effects on the sleep of the patient and that this effect was greater to the one when each of these two hypnotic agents and/or sleep aids are taken separately.

### DETAILED DESCRIPTION OF THE INVENTION

The terms as used herein have the following meanings:

As used herein, the expression “C₁₋₂₀ alkyl” includes methyl, ethyl, and straight-chained or branched propyl, butyl, pentyl, hexyl, heptyl, octyl, nonyl, decyl, etc. Particular alkyl groups are without any limitation, methyl, ethyl, n-propyl, isopropyl, n-butyl, isobutyl, tert-butyl, n-pentyl, amyl, isoamyl, n-hexyl, etc.

As used herein, “patient” means a warm blooded animal, such as for example rat, mice, dogs, cats, guinea pigs, and primates such as humans.

As used herein, the expression “pharmaceutically acceptable carrier” means a non-toxic solvent, dispersant, excipient, adjuvant, or other material which is mixed with the compound of the present invention in order to permit the formation of a pharmaceutical composition, i.e., a dosage form capable of administration to the patient. One example of such a carrier is pharmaceutically acceptable oil typically used for parenteral administration.

The term “pharmaceutically acceptable salts” as used herein means that the salts of the compounds of the present invention can be used in medicinal preparations. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, hydrobromic acid, sulfuric acid, methanesulfonic acid, 2-hydroxyethanesulfonic acid, p-toluenesulfonic acid, fumaric acid, maleic acid, hydroxymaleic acid, malic acid, ascorbic acid, succinic acid, glutaric acid, acetic acid, salicylic acid, cinnamic acid, 2-phenoxybenzoic acid, hydroxybenzoic acid, phenylacetic acid, benzoic acid, oxalic acid, citric acid, tartaric acid, glycolic acid, lactic acid, pyruvic acid, malonic acid, carbonic acid or phosphoric acid. The acid metal salts such as sodium monohydrogen orthophosphate and potassium hydrogen sulfate can also be formed. Also, the salts so formed may present either as mono- or di-acid salts and can exist substantially anhydrous or can be hydrated. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts, and salts formed with suitable organic ligands, e.g. quaternary ammonium salts.

As used herein, the term “prodrug” shall have the generally accepted meaning in the art. One such definition includes a pharmacologically inactive chemical entity that when metabolized or chemically transformed by a biological system such as a mammalian system is converted into a pharmacologically active substance.

The expression “stereoisomers” is a general term used for all isomers of the individual molecules that differ only in the orientation of their atoms in space. Typically it includes mirror image isomers that are usually formed due to at least one asymmetric center (enantiomers). Where the compounds according to the invention possess two or more asymmetric centers, they may additionally exist as diastereoisomers, also certain individual molecules may exist as geometric isomers (cis/trans). Similarly, certain compounds of this invention may exist in a mixture of two or more structurally distinct forms that are in rapid equilibrium, commonly known as tautomers. Representative examples of tautomers include keto-enol tautomers, phenol-keto tautomers, nitroso-oxime tautomers, imine-enamine tautomers, etc. It is to be understood that all such isomers and mixtures thereof in any proportion are encompassed within the scope of the present invention.

The term “solvate” as used herein means that an aggregate that consists of a solute ion or molecule with one or more solvent molecules. Similarly, a “hydrate” means that a solute ion or molecule with one or more water molecules.

In a broad sense, the term “substituted” is contemplated to include all permissible substituents of organic compounds. In a few of the specific embodiments as disclosed herein, the term “substituted” means substituted with one or more substituents independently selected from the group consisting of C₁₋₆alkyl, C₂₋₆alkenyl, C₁₋₆perfluoroalkyl, phenyl, hydroxy, —CO₂H, an ester, an amide, C₁-C₆alkoxy, C₁-C₆thioalkyl, C₁-C₆ perfluoroalkoxy, —NH₂, Cl, Br, I, F, —NH-lower alkyl, and —N(lower alkyl)₂. However, any of the other suitable substituents known to one skilled in the art can also be used in these embodiments.

“Therapeutically effective amount” means an amount of the combination or composition which is effective in treating the named disease, disorder or condition.

“Administering” comprises administration via any appropriate route such as oral, sublingual, buccal, transdermal, inhalation, rectal or injection (including intramuscular, intravenous, subcutaneous, etc.), or any other appropriate method of providing the combination or the composition to the patient.

The term “treating” refers to:
(i) preventing a disease, disorder or condition from occurring in a patient that may be predisposed to the disease, disorder and/or condition, but has not yet been diagnosed as having it;
(ii) inhibiting the disease, disorder or condition, i.e., arresting its development; and
(iii) relieving the disease, disorder or condition, i.e., causing regression of the disease, disorder and/or condition.

The term “short acting hypnotic agent” is referred to a compound and/or agent that is capable of inducing sleep, i.e., the entry time into the sleep phase.

The term “long acting hypnotic agent” is referred to a compound or agent that is mainly a sleep inducer but may also be capable of improving sleep quality and/or maintenance in a patient.

The term “sleep aid” is referred to a compound or agent that is mainly used to improve sleep quality and/or sleep maintenance in a patient, in particular the deep sleep phases.

The term “restorative sleep” means sleep which produces a rested state upon waking.

The term “sleep disorder” as used herein shall mean all of the description as delineated in the Diagnostic and Statistical Manual of Mental Disorders, 4^{th} Edition (1994), hereafter referred to as DSM-IV, published by the American Psychiatric Association. Specific sleep disorders that can be treated in accordance with this invention include without any limitation insomnia, primary insomnia, sleep maintenance insomnia, insomnia related to another mental disorder, substance induced insomnia and obstructive sleep apnea. Further description and discussion of sleep disorders are found in the International Classification of Sleep Disorders: Diagnostic and Coding Manual (1990), published by the American Sleep Disorders Association.

The term “insomnia” as used herein includes all sleep disorders, which are not caused due to other factors such as mental disorders, other medical conditions and substance induced sleep disorders. Insomnia as used herein shall also mean primary sleep disorders as defined in DSM-IV, which includes two sub-categories, namely, dyssomnias and parasomnias.

The term “primary insomnia” shall mean all of the definitions provided in DSM-UV. In addition, “primary insomnia” as used herein also includes “sleep maintenance insomnia.” The DSM-IV lists the diagnostic criteria for primary insomnia as follows:
A. The predominant complaint is difficulty initiating or maintaining sleep, or nonrestorative sleep, for at least one month.
B. The sleep disturbance (or associated day time fatigue) causes clinically significant distress or impairment in social, occupational, or other important areas of functioning.
C. The sleep disturbance does not occur exclusively during the course of narcolepsy, breathing-related sleep disorder, circadian rhythm sleep disorder, or a parasomnia.
D. The disturbance does not occur exclusively during the course of another mental disorder (e.g., major depressive disorder, generalized anxiety disorder, a delirium).
E. The disturbance is not due to the direct physiological effects of a substance (e.g., a drug of abuse, a medication) or a general medical condition.

The term “sleep disorder related to another mental disorder” as used herein includes both insomnia and hypersomnia related to another mental disorder. The DSM-IV lists the diagnostic criteria for insomnia related to another mental disorder as follows:
A. The predominant complaint is difficulty initiating or maintaining sleep, or nonrestorative sleep, for at least one month that is associated with daytime fatigue or impaired daytime functioning.
B. The sleep disturbance (or daytime sequelae) causes clinically significant distress or impairment in social, occupational, or other important areas of functioning.
C. The insomnia is judged to be related to another axis I or axis II disorder (e.g., major depressive disorder, generalized anxiety disorder, adjustment disorder with anxiety, schizophrenia, etc.), but is sufficiently severe to warrant independent clinical attention.
D. The disturbance is not better accounted for by another sleep disorder (e.g., narcolepsy, breathing-related sleep disorder, a parasomnia).
E. The disturbance is not due to the direct physiological effects of a substance (e.g., a drug of abuse, a medication) or a general medical condition.

Similarly, the DSM-IV lists the diagnostic criteria for hypersomnia related to another mental disorder as follows:
A. The predominant complaint is excessive sleepiness for at least one month as evidenced by either prolonged sleep episodes or daytime sleep episodes that occur almost daily.
B. The excessive sleepiness causes clinically significant distress or impairment in social, occupational, or other important areas of functioning.
C. The hypersomnia is judged to be related to another axis I or axis II disorder (e.g., major depressive disorder, dysthymic disorder, schizophrenia, etc.), but is sufficiently severe to warrant independent clinical attention.
D. The disturbance is not better accounted for by another sleep disorder (e.g., narcolepsy, breathing-related sleep disorder, a parasomnia) or by an inadequate amount of sleep.
E. The disturbance is not due to the direct physiological effects of a substance (e.g., a drug of abuse, a medication) or a general medical condition.

The term “substance induced sleep disorder” as used herein means a prominent disturbance in sleep that is sufficiently severe to warrant independent clinical attention and is judged to be due to the direct physiological effects of a substance (i.e., a drug of abuse, a medication, or toxin exposure). Specific examples of drug of abuse, a medication or toxin exposure as referred to herein include without any limitations caffeine, alcohol, amphetamine, opioids, sedatives, hypnotics, anxiolytics, and the like. The DSM-IV lists the diagnostic criteria for substance induced sleep disorder as follows:
A. A prominent disturbance in sleep that is sufficiently severe to warrant independent clinical attention.
B. There is evidence from the history, physical examination, or laboratory findings of either (1) or (2): (1) the symptoms in criterion A developed during, or within a month of, substance intoxication or withdrawal; (2) medication use is etiologically related to the sleep disturbance.
C. The disturbance is not better accounted for by a sleep disorder that is not substance induced. Evidence that the symptoms are better accounted for by a sleep disorder that is not substance induced might include the following: the symptoms precede the onset of the substance use (or medication use); the symptoms persist for a substantial period of time (e.g., about a month) after the cessation of acute withdrawal or severe intoxication, or are substantially in excess of what would be expected given the type or amount of the substance used or the duration of use; or there is evidence that suggests the existence of an independent non-substance-induced sleep disorder (e.g., a history of recurrent non-substance-related episodes).
D. The disturbance does not occur exclusively during the course of a delirium.
E. The sleep disturbance causes clinically significant distress or impairment in social, occupational, or other important areas of functioning.

As used herein “withdrawal” refers to a syndrome characterized by untoward physical changes that occur following cessation of or reduction in substance use, or administration of a pharmacologic antagonist (or medication).

The term “obstructive sleep apnea” as used herein is breathing related sleep disorder as defined in DSM-IV. It is also referred to as upper airway resistance syndrome and generally involves repeated episodes of upper-airway obstruction during sleep and is normally characterized by loud snores or brief gasps that alternate with episodes of silence. The DSM-IV lists the diagnostic criteria for breathing related sleep disorder as follows:
A. Sleep disruption, leading to excessive sleepiness or insomnia, that is judged to be due to a sleep-related breathing condition (e.g., obstructive sleep or central sleep apnea syndrome or central alveolar hypoventilation syndrome).

B. The disturbance is not better accounted for by another mental disorder and is not due to the direct physiological effects of a substance (e.g., a drug of abuse, a medication) or another general medical condition (other than a breathing related disorder).

Subjective and Objective Determinations of Sleep Disorders: There are a number of ways to determine whether the onset, duration or quality of sleep (e.g. non-restorative or restorative sleep) is impaired or improved. One method is a subjective determination of the patient, e.g., do they feel drowsy or rested upon waking. Other methods involve the observation of the patient by another during sleep, e.g., how long it takes the patient to fall asleep, how many times does the patient wake up during the night, how restless is the patient during sleep, etc. Another method is to objectively measure the stages of sleep.

Polysomnography is the monitoring of multiple electrophysiological parameters during sleep and generally includes measurement of EEG activity, electroculographic activity and electromyographic activity, as well as other measurements. These results, along with observations, can measure not only sleep latency (the amount of time required to fall asleep), but also sleep continuity (overall balance of sleep and wakefulness) which may be an indication of the quality of sleep.

There are five distinct sleep stages which can be measured by polysomnogrpahy: rapid eye movement (REM) sleep and four stages of no-rapid eye movement (NREM) sleep (stages 1, 2, 3 and 4). Stage 1 NREM sleep is a transition from wakefulness to sleep and occupies about 5% of time spent asleep in healthy adults. Stage 2 NREM sleep, which is characterized by specific EEG waveforms (sleep spindles and K complexes), occupies about 50% of time spent asleep. Stages 3 and 4 NREM sleep (also known collectively as slow-wave sleep) are the deepest levels of sleep and occupy about 10-20% of sleep time. REM sleep, during which the majority of typical story like dreams occur, occupies about 20-25% of total sleep.

These sleep stages have a characteristic temporal organization across the night. NREM stages 3 and 4 tend to occur in the first one-third to one-half of the night and increase in duration in response to sleep deprivation. REM sleep occurs cyclically through the night. Alternating with NREM sleep about every 80-100 minutes. REM sleep periods increase in duration toward the morning. Human sleep also varies characteristically across the life span. After relative stability with large amounts of slow-wave sleep in childhood and early adolescence, sleep continuity and depth deteriorate across the adult age range. This deterioration is reflected by increased wakefulness and stage 1 sleep and decreased stages 3 and 4 sleep.

Thus in accordance with this invention there is provided a combination of two hypnotic agents, or at least one hypnotic agent and at least one sleep aid. The combination of the invention comprises at least a short or long-acting hypnotic agent and a sleep aid. In accordance with this aspect of the invention, the short or long-acting hypnotic agent is present in a galenic formulation adapted to an immediate or delayed release, and the sleep aid is present in the form of a galenic formulation adapted to an immediate-release.

More particularly, the present invention provides a combination of at least one short acting hypnotic agent with Compound A or its prodrug or a pharmaceutically acceptable salt thereof, wherein the prodrug is of the Formula II;

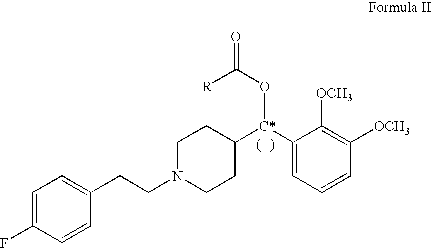

wherein R is C₁-C₂₀ alkyl.

The combination of a short and/or long-acting hypnotic agent with a sleep aid allows to obtain beneficial effects on the sleep of the patient and that this effect is greater to the one when each of these two hypnotic agents are taken separately.

In accordance with the first aspect of the invention, the short-acting hypnotic agent and Compound A are released immediately. The two agents then appear in the plasma according to their respective pharmacokinetic characteristics. Generally, the short-acting hypnotic agent appears in the plasma before the long-acting hypnotic agent. Further, in this aspect of the invention, each agent develops its mechanism of action independent of each other, providing a synergistic effect between the two agents.

In yet another aspect of the invention, the short-acting hypnotic agent is released with a delay and the sleep aid, such as Compound A, is released immediately. According to this aspect of the invention, the action of the short-acting hypnotic agent is increased with increasing residence time in the plasma. Thus, the two agents can act at the same time, also with a synergistic effect.

Examples of short-acting hypnotic agents useable within the framework of the invention are in particular the modulators of the GABA-A receptors, the benzodiazepines, the melatonin derivatives, the agonists of the melatonin receptors. For example, the short-acting hypnotic agent can be chosen from among, in particular, zolpidem, zopiclone, eszopiclone, zaleplon, melatonin, ramelteon, triazolam, etizolam, brotizolam and indiplon, as well as derivatives and/or mixtures thereof.

Examples of long-acting hypnotic agents and/or the sleep aids useable within the framework of the invention are in particular the antagonists of the 5HT2A receptors, the modulators of the GABA-A receptors, benzodiazepines and the modulators of calcium ions. For example, the long-acting hypnotic agent and/or the sleep aids can be chosen from among, in particular, the Compound A or its prodrugs, temazepam, clonazepam, gaboxadol, pregabaline, as well as derivatives and/or mixtures thereof.

The short or long-acting hypnotic agents and/or the sleep aids described above can comprise one or more asymmetric carbon atoms. They can thus exist in the form of enantiomers or diastereoisomers. These enantiomers or diastereoisomers, as well as mixtures thereof, including the racemic mixtures, are part of the invention.

The short or long-acting hypnotic agents and/or sleep aids described above can also exist in the form of free bases or acids as well as their pharmaceutically acceptable salts. Such salts are also part of the invention. These salts can be prepared with pharmaceutically acceptable acids or bases following the procedures well known in the art.

The short or long-acting hypnotic agents and/or sleep aids described above can also exist in the form of hydrates or solvates, i.e., in a form of associations or combinations with one or more molecules of water or a solvent. Such hydrates and solvates are also part of the invention.

According to one embodiment of the invention, the combination comprises zolpidem hemitartarate as short-acting hypnotic agent and the Compound A as a sleep aid.

The Compound A may be synthesized by methods known in the art, such as one previously described in U.S. Pat. No. 5,134,149, incorporated herein by reference,

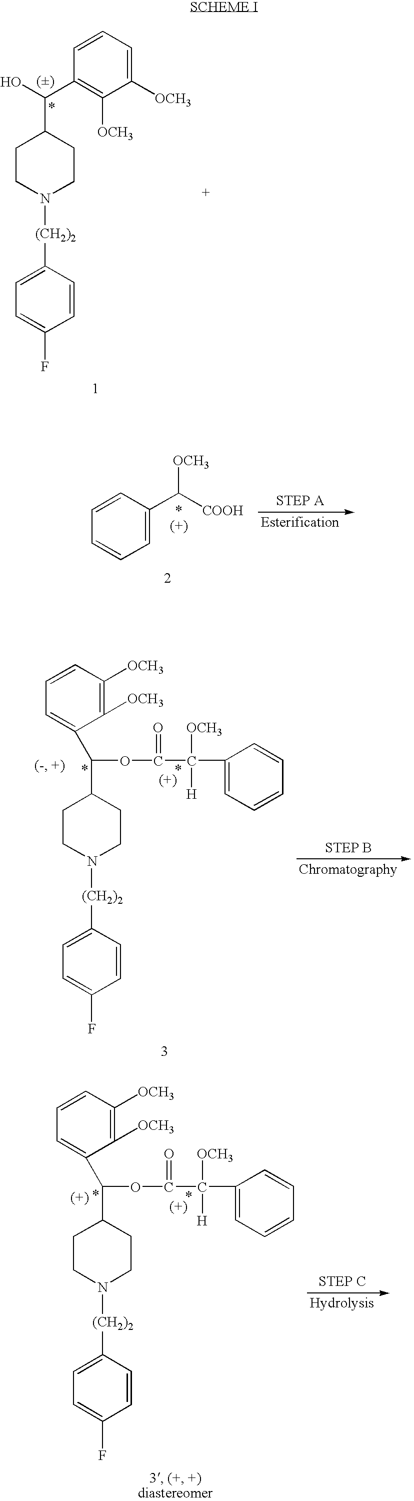

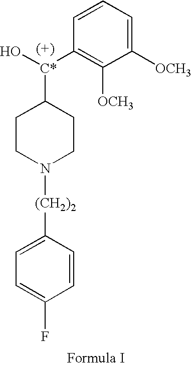

In Step A of Reaction Scheme I, an esterification reaction is carried out between racemic α-(2,3-dimethoxyphenyl)-1-[2-(4-fluorophenyl)ethyl]-4-piperidinemethanol (structure 1) and the (+)-isomer of α-methoxyphenylacetic acid (structure 2). This esterification produces the diastereomeric mixture identified as structure 3. These diastereomers are subjected to silica gel chromatography which separates the two diastereomers, thereby isolating the (+,+) diastereomer as is depicted in Step B. In Step C, the (+,+) diastereomer is hydrolyzed which produces the (+)-isomer of α-(2,3-dimethoxy-phenyl)-1-[2-(4-fluorophenyl)ethyl]-4-piperidinemethanol.

The esterification reaction can be carried out using techniques known in the art. Typically approximately equivalent amounts of racemic α-(2,3-dimethoxyphenyl)-1-[2-(4-fluorophenyl)ethyl]-4-piperidinemethanol and the (+)-isomer of α-methoxyphenylacetic acid are contacted in an organic solvent such as methylene chloride, THF, chloroform, toluene and heated to reflux for a period of time ranging from 5 to 24 hours. The esterification is typically carried out in the presence of an equivalent amount of dicyclohexylcarbodiimide and a catalytic amount of 4-dimethylaminopyridine. The resulting diastereomers can be isolated by filtration of the dicyclohexylurea and evaporation of the filtrate.

The diastereomers are then subjected to silica gel chromatography which separates the (+,+) and the (−,+) diastereomers. This chromatographic separation may be carried out as is known in the art. A 1:1 mixture of hexane and ethyl acetate is one suitable eluent.

The resulting (+,+) diastereomer is then subjected to a hydrolysis reaction which produces the (+)-isomer of α-(2,3-dimethoxyphenyl)-1-[2-(4-fluorophenyl)ethyl]-4-piperidinemethanol. The hydrolysis is carried out by contacting the diastereomer with an excess of a base such as potassium carbonate in an aqueous alcoholic solution. The hydrolysis is carried out at a temperature of about 15 to 30° C. for a period of time ranging from 2 to 24 hours. The resulting (+)-isomer of α-(2,3-dimethoxyphenyl)-1-[2-(4-fluorophenyl)ethyl]-4-piperidinemethanol may then be recovered by dilution with water and extraction with methylene chloride. It is then purified by recrystallization from a solvent system such as cyclohexane/hexane or ethyl acetate/hexane.

Methods for producing the starting materials of Reaction Scheme I are known in the art. For example, U.S. Pat. No. 4,783,471 teaches how to prepare racemic α-(2,3-dimethoxyphenyl)-1-[2-(4-fluorophenyl)ethyl]-4-piperidinemethanol. This patent is hereby incorporated by reference. Examples No. 1 and 2 of this application also teach suitable methods. Alternatively, racemic α-(2,3-dimethoxyphenyl)-1-[2-(4-fluorophenyl)ethyl]-4-piperidinemethanol can be prepared in the following manner. Initially 4-hydroxypiperidine is subjected to an N-alkylation reaction with p-fluorophenylethyl bromide which produces 4-hydroxy-1-[2-(4-fluorophenyl)ethyl]-piperidine. This compound is brominated with Ph₃P.Br₂ which produces 4-bromo-1-[2-(4-fluorophenyl)ethyl]piperidine. This compound is contacted with Mg thereby forming a Grignard Reagent which is then reacted with 2,3-dimethoxybenzaldehyde which produces the desired product (±)-α-(2,3-dimethoxyphenyl)-1-[2-(4-fluorophenyl)ethyl]-4-piperidinemethanol. The (+)-isomer of α-methoxyphenylacetic acid is known in the art. The prodrugs of Compound A can be prepared following the procedures described in the art. For instance U.S. Pat. No. 6,028,083 discloses various procedures to prepare a few of the prodrugs of Compound A.

The short acting hypnotic agents as described herein can also be prepared by various known procedures described in the art. For example, preparation of zolpidem is described in U.S. Pat. No. 4,382,938, which is incorporated herein by reference.

According to another aspect, the invention concerns pharmaceutical compositions comprising, as active principle, at least one short-acting hypnotic agent and at least one long-acting hypnotic agent and/or a sleep aid. The pharmaceutical compositions of the invention comprise an effective dose of at least one short-acting hypnotic agent and at least one long-acting hypnotic agent and/or a sleep aid, or a pharmaceutically acceptable salt of these agents, a hydrate or solvate of said agents, as well as at least a pharmaceutically acceptable excipient.

The excipients are chosen according to the desired pharmaceutical form and administration mode, from among the usual excipients known to a person skilled in the art. The short or long-acting hypnotic agents and the sleep aids can be chosen from among the ones described above.

The unit-dose packages of appropriate administration comprise the forms: via oral administration, such as tablets, particularly multi-layer tablets, coated tablets, tablets with a core, soft or hard capsules, powders, granules and oral solutions or suspensions, sublingual or by mouth administration forms.

In another embodiment of this invention, the long-acting hypnotic agent and/or the sleep aid such as Compound A and the short-acting hypnotic agents present in the composition according to the invention, are released immediately.

In yet another embodiment of this invention, the long-acting hypnotic agent and/or the sleep aid such as Compound A present in the composition according to the invention is immediately released and the short-acting hypnotic agent is released with a delay.

The immediate-release entity can be a unit with immediate-release of a pharmaceutical product such as, for example, a tablet or a capsule with immediate-release, or several of these units in the form of tablet formulated in a capsule; the immediate-release system of one tablet; an immediate-release layer incorporated in a multi-layer tablet; one or more coating layers in a tablet or pellet.

The delayed release entity can be a unit with delayed release of a pharmaceutical product such as, for example, a delayed-release tablet or capsule; or several of these units formulated in a capsule; a delayed-release layer incorporated in a multi-layer tablet; a delayed-release core or a coating layer incorporated in a tablet with several coats; delayed-release pellets inside a disintegrating tablet.

The long-acting hypnotic agent and/or the sleep aid, and the short-acting hypnotic agent can be formulated according to the invention in one single pharmaceutical composition, or, alternatively, in separate pharmaceutical compositions for a simultaneous, separate, or sequential administration.

Orally, the dose of active principle present in a composition according to the invention varies from about 0.1 to about 30 mg of long-acting hypnotic agent and about 0.1 to about 30 mg of short-acting hypnotic agent

For example, a composition according to the invention contains about 0.2 to about 15 mg, in particular from 1 to 10 mg Compound A, and about 0.2 to about 20 mg, in particular from 1 to 10 mg zolpidem in base form.

Particular cases can exist where higher or lower dosages are appropriate; such dosages are not outside the scope of the invention. According to the usual practice, the appropriate dosage for each patient is determined by the physician, depending on the mode of administration, the weight, and the response of said patient.

In an embodiment of the compositions according to the invention consists in a capsule comprising one or more immediate-release tablets containing the short-acting hypnotic agent and one or more immediate-release tablets containing the long-acting hypnotic agent and/or the sleep aid such as Compound A.

In another embodiment of the compositions according to the invention consists in a capsule comprising one or more delayed-release tablets containing the short-acting hypnotic agent and one or more immediate-release tablets containing the long-acting hypnotic agent and/or the sleep aid such as Compound A.

Another embodiment of the compositions according to the invention consists in a capsule comprising a mixture of immediate-release pellets of the short-acting hypnotic agent and of immediate-release pellets of the long-acting hypnotic agent and/or the sleep aid such as Compound A.

Yet another embodiment of the compositions according to the invention consists in a capsule comprising a mixture of immediate-release pellets of the short-acting hypnotic agent and of immediate-release pellets of the long-acting hypnotic agent and/or the sleep aid such as Compound A.

In a further embodiment of the compositions according to the invention consists in a tablet comprising immediate-release pellets of the short-acting hypnotic agent and the long-acting hypnotic agent and/or the sleep aid such as Compound A.

Yet another embodiment of the compositions according to the invention consists in a tablet comprising delayed-release pellets of the short-acting hypnotic agent and of immediate-release pellets of the long-acting hypnotic agent and/or the sleep aid such as Compound A.

Another embodiment of the compositions according to the invention consists in an enteric-coated, delayed-release tablet comprising immediate-release pellets of the long-acting hypnotic agent and/or a sleep aid such as Compound A, and of immediate-release pellets of the short-acting hypnotic agent.

Another embodiment of the compositions according to the invention consists in a dry-coated tablet, characterized in that it comprises a delayed-release inner core containing the long-acting hypnotic agent and/or the sleep aid such as Compound A, and in that the immediate-releasing coating layer contains the long-acting hypnotic agent and/or the sleep aid such as Compound A.

In another aspect of this invention, a specific disease, a disorder or a condition that can be treated with the combination and/or the pharmaceutical composition comprising the combination of this invention include, without any limitation a wide variety of sleep disorders. As already noted hereinabove, specific sleep disorders that can be treated in accordance with this invention include without any limitation insomnia, primary insomnia, sleep maintenance insomnia, insomnia related to another mental disorder, substance induced insomnia and obstructive sleep apnea.

The compositions according to the invention can be prepared according to the methods known by a person skilled in the art.

Thus, the capsules containing one or more reduced-size, immediate-release tablets containing the long-acting hypnotic agent and/or the sleep aid, and one or more reduced-size, immediate-release tablets containing the short-acting hypnotic agent can be prepared as follows.

The immediate-release tablets can be prepared with direct compression of active principle mixtures in the base form or salts with diluents such as microcrystalline cellulose, mannitol, sorbitol, lactose. Other excipients, such as disintegrators or lubricants, can be added. The choice between these functional excipients, as well as these diluents, is well known by a person skilled in the art.

According to another embodiment, tablets can be prepared by granulation with water or solvents of a mixture of one or more of the active principles mixed with diluents, appropriate disintegrating agents and polymers, then calibration and drying of the obtained pellet, addition of lubricating agent, followed by a compression with a compression machine. Various methods of tablet making are generally described in literature, such as, for example, B. B. Sheth, F. J. Bandelin, R. J F. Shangraw, Compressed tablets, in Pharmaceutical dosage forms: Tablets, Vol 1, published by H. A. Lieberman and L Lachman, Dekker N, Y. (1980).

Capsules containing one or more reduced-size, immediate-release tablets containing the long-acting hypnotic agent, and/or a sleep aid and one or more reduced-size, delayed-release tablets containing the short-acting hypnotic agent can be prepared following the known procedures in the art.

Delayed-release tablets containing the short acting hypnotic agent can be prepared by coating the immediate-release tablets, such as described above, with a polymer coating having a limited diffusion. Such polymers can be chosen among ethylcellulose copolymers as well as methyl methacrylate polymers, such as commercialized products named Eudragit TM RS®, Eudragit TM RL®, Eudragit TM NE®, all of which are commercially available from Rohm Pharma.

Coating methods can consist in pulverization of a polymer solution on the tablets, in a coating machine or a fluidized bed device. The solvent that can be employed is either organic or aqueous, depending on the nature of the polymer used. Coating methods are described, in particular in J. M. Bakan, Microencapsulation, in L. Lachman, H. Lieberman and J. L. Kanig (Eds), The Theory and Practice of Industrial Pharmacy, Lea & Febinger, Philadelphia, USA, 1986; J. M. Mc Ginity, Aqueous Polymer Coatings for Pharmaceutical Dosage Forms, Dekker N.Y., 1989.

Delayed-release tablets can also be prepared with the incorporation of excipients forming the matrix in the formulation, with no disintegrating agent. Examples of excipients, forming the matrix are the hydrophilic polymers, in particular hydroxypropylmethylcellulose, hydroxymethylcellulose, hydroxyethylcellulose, which expand when in contact with aqueous liquids and which can control the release of the active principle through the expanded polymeric network. Such excipients are used in a quantity in percentage weight of about 10% to about 40% of the total weight of the tablet.

Delayed-release tablets can also be formulated, in the case of basic active principles, with a pharmaceutically acceptable organic acid, chosen among those indicated hereafter, in order to maintain its dissolution in the neutral pH conditions in the small intestine. Examples of organic acids useable are among maleic, tartaric, malic, fumaric, lactic, citric, adipic and succinic acid.

Capsules containing a mixture of immediate-release pellets of the long and short-acting hypnotic agent and/or a sleep aid can be prepared as follows. Immediate-release pellets of the long and short-acting hypnotic agent and/or a sleep aid can be prepared by precipitating the active principle in suspension in water with, for example, hydroxypropylmethylcellulose or in an organic solvent such as ethanol or another appropriate polymer acting as a binder on a spherical granule. A coating device with fluidized bed is generally used. Particles can be agglomerated in order to form spherical granules or pellets, in a high-speed granulator-mixer or a rotary agglomerator with fluidized bed. Such methods are described in K. W. Olson, A. M. Mehta, Int. J. Phar. Tech & Prod. Mfr. 6 18-24, 1985. Pellets can generally be prepared by mass extrusion or by melting followed by spheronization, as described, for example, in C. Vervaet, L. Baert & J. P. Remon, Int. J. Pharm. 116 (1995)131-146.

The excipients used are typically those having good plastic qualities such as microcrystalline cellulose, mannitol. Small quantities of binder are generally added. Surfactant agents, such as sodium dodecyl sulfate can also be incorporated in order to facilitate the extrusion.

Capsules containing a mixture of immediate-release pellets of long-acting hypnotic agent and/or the sleep aid such as Compound A, and delayed-release pellets of short-acting hypnotic agent can be prepared as follows. Immediate-release pellets can be prepared as described above. Delayed-release pellets can contain, in the case of basic active principles, a pharmaceutically acceptable organic acid or an acid salt of such organic acid, for maintaining the local pH inside the pellet during its dissolution under neutral pH in the small intestine.

Alternately, pellets can be coated with pH sensitive membrane, containing a polymer soluble under neutral pH and impermeable to an acid pH, such as, for example, the product Eudragit TM S®, which allows a permeation of the active principle at a pH higher than about 5, for compensating the reduced solubility of the active principle at low pH levels.

Tablets containing several immediate-release pellets of long-acting hypnotic agent and/or a sleep aid and short-acting hypnotic agent can be prepared as follows. The different pellets can be immersed in a matrix where the matrix itself can contain one of the hypnotic agents. Then tablets disintegrate when they are in contact with a fluid, releasing quickly the active principle, or immediate-release pellets, or from the coating of immediate-release pellets.

Tablets containing one or several immediate-release pellets of long-acting hypnotic agent and/or a sleep aid and one or several delayed-release pellets of short-acting hypnotic agent can be prepared as follows.

1) The tablet can consist in a mixture of immediate-release pellets and delayed-release pellets containing the active principle, immersed in a matrix which does not contain an active principle.
2) Alternatively, pellets containing the two hypnotic agents and/or sleep aids can be immersed in a matrix containing itself one of the two therapeutic agents.

According to another embodiment of this invention, delayed-release pellets can be coated with a layer containing the active principle and excipients, allowing an immediate-release from this coating layer, immersed in a matrix with no active principle. The matrix surrounding the pellets is formulated in order that the compression in tablets does not interfere with the membrane integrity surrounding the pellets. Tablet disintegrates when it is in contact with a fluid, releasing quickly the long-acting hypnotic agent and/or a sleep aid, from the matrix or immediate-release pellets, or from the coatings of immediate-release pellets and by releasing then the short-acting hypnotic agent, from delayed-release pellets.

The pharmaceutical composition of the invention can also be found in the form of a multilayer tablet. Such a multilayer tablet comprises:

One or several layer with immediate-release, each one containing a dose of long-acting hypnotic agent and/or a sleep aid, and eventually a dose of short-acting hypnotic agent;

One or more layers with delayed release, each one containing a dose of short-acting hypnotic agent; and

Eventually a supplementary layer which does not contain any active principle but contains hydrophilic polymers such as the cellulose derivative, for example, hydroxypropylcellulose, hydroxyethylcellulose, hydroxymethylcellulose, or soluble diluents, such as, lactose, sorbitol, mannitol, one or more other hydrophilic polymers and/or one or more other soluble excipients, this layer modulating the active principle release from the delayed release layer. Each layer contains eventually other excipients, in order to allow a good compression, lubrication, and binder of the tablet.

Another embodiment of this invention consists in a core containing the short-acting hypnotic agent, eventually with a pharmaceutically acceptable organic acid. The core is coated with a polymer layer containing the long-acting hypnotic agent and/or a sleep aid that is quickly or immediately released in contact with fluids, while the short-acting hypnotic agent is released from the core. Eventually, the core and the coating layer can be formulated in order to allow a release in the colon. Each constituent of the multiple coated tablet can contain other excipients, to allow a good compression, lubrication and binder. Preparation processes of multiplayer tablets and multiple coating tablets are described in particular in W. C. Gunsel, Compression coated and layer tablets in pharmaceutical dosage forms: tablets, Vol 1, published by H. A. Lieberman and L. Lachman, Dekker N.Y. (1980).

This invention is further illustrated by the following examples which are provided for illustration purposes and in no way limit the scope of the present invention.

Examples 1, 2 and 3 show one method of making the Compound A. Example 4 shows how to use the combination of this invention and Examples 5 to 15 provide methods for the preparations of the pharmaceutical compositions of the combination of the invention with Compound A and a short acting hypnotic.

As used herein, “DMF” means dimethylformamide; “CH₂Cl₂” means methylene chloride or dichloromethane; “EtOAc” means ethyl acetate; “THF” means tetrahydrofuran; “MeOH” means methanol or methyl alcohol; “K₂CO₃” means potassium carbonate; “NaHCO₃” means sodium bicarbonate; “MgSO₄” means magnesium sulfate; “POCl₃” means phosphorus oxychloride; “NH₄OH” means ammonium hydroxide; “NH₄Cl” means ammonium chloride; “DIBAL-H” means diisobutylaluminum hydride; “HCl” means hydrochloric acid; “NaOH” means sodium hydroxide; “n-BuLi” means n-butyl lithium; “NaBH₄” means sodium borohydride; “brine” means saturated aqueous sodium chloride solution; “TLC” means thin layer chromatography; “Rf” means retention factor; “H₂O” means water; and “N₂” means nitrogen.

### EXAMPLE 1

Example 1, Steps A-D, demonstrates the preparation of the starting material (±)-α-(2,3-dimethoxyphenyl)-1-[2-(4-fluorophenyl)ethyl]-4-piperidinemethanol, structure 1.

### A) 1-[2-(4-Fluorophenyl)ethyl]-4-piperidinecarboxamide

A solution of isonipectoamide (10.9 g, 85.0 mmol), 2-(4-fluorophenyl)ethyl bromide (15.7 g, 77.3 mmol), and K₂CO₃ (2.3 g, 167 mmol) was prepared in DMF (280 mL) and stirred under argon at 90-95° C. overnight. The cooled solution was concentrated to a white oily solid. The solid was partitioned between water and CH₂Cl₂. The layers were separated and the aqueous layer was extracted with CH₂Cl₂. The combined organic layers were washed 2× with water, dried (MgSO₄), filtered, and evaporated to an oily solid. The solid was recrystallized from EtOAc to afford 1-[2-(4-fluorophenyl)ethyl]-4-piperidinecarboxamide as a white powder, m.p. 177-178° C. (decomp.). Anal. Calcd. for C₁₄H₁₉FN₂O: C, 67.18; H, 7.65: N, 11.19. Found: C, 67.25; H, 7.67; N, 11.13.

### B) 4-Cyano-1-[2-(4-fluorophenyl)ethyl]piperidine

To stirred POCl₃ (25 ml, 41.12 g, 268 mmol) and sodium chloride (5.1 g, 87.3 mmol) was added 1-[2-(4-fluorophenyl)ethyl]-4-piperidinecarboxamide (8.9 g, 35.6 mmol) portionwise. After complete addition, the solution was refluxed for 2 hours. The cooled solution was poured into dilute NH₄OH to destroy the POCl₃. The aqueous solution was cooled to 0° C., then extracted 2× with CH₂Cl₂. The combined organic layers were dried (MgSO₄), filtered, and evaporated to afford 8.1 g of an oily solid. The solid was distilled, (b.p. 150° C., 0.1 mm Hg), to afford a clear, colorless oil that solidified. This material was crystallized from hexane to afford 4-cyano-1-[2-(4-fluorophenyl)ethyl]piperidine as white needles, m.p. 47-48° C. Anal. Calcd. for C₁₄H₁₇FN₂: C, 72.39; H, 7.38; N, 12.06. Found: C, 72.62; H, 7.49; N, 12.12.

### C) 1-[2-(4-Fluorophenyl)ethyl]-4-piperidinecarboxaldehyde

To a stirred solution of 4-cyano-1-[2-(4-fluorophenyl)-ethyl]piperidine (1.00 g, 4.3 mmol) in THF (20 mL) under argon at 0° C. was added DIBAL-H (4.6 mL of a 1.0 M solution in THF, 4.6 mmol) via syringe. After stirring overnight at room temperature, 10% aqueous HCl (25 mL) was added and the solution was stirred for 3 hours. The entire mixture was then poured into 10% aqueous NaOH (50 mL), then extracted 2× with ether. The combined organic layers were washed with brine, dried (MgSO₄), filtered, and evaporated to afford a pale yellow oil. The oil was chromatographed on silica gel, eluting with EtOAc. The appropriate fractions were combined and evaporated to afford an oil. This oil was distilled (b.p. 166° C., 0.05 mm Hg) to afford 1-[2-(4-fluorophenyl)ethyl]-4-piperidinecarboxaldehyde, obtained as a colorless oil. Anal. Calcd. for C₁₄H₁₈FNO: C, 71.46; H, 7.71; N, 5.95. Found: C, 71.08; H, 7.81; N, 5.86.

### D) (±)-α-(2,3-Dimethoxyphenyl)-1-[2-(4-fluorophenyl)ethyl]-4-piperidinemethanol

To a stirred solution of veratrole (0.93 g, 6.7 mmol) in THF (20 mL) under argon at 0° C. was added n-BuLi (2.7 mL of a 2.5 M solution in hexane, 6.75 mmol). After stirring 2.5 h, the solution was cooled to −78° C. and treated with 1-[2-(4-fluorophenyl)ethyl]-4-piperidinecarboxaldehyde (1.30 g, 5.5 mmol) in THF (25 mL) via an additional funnel. The cooling bath was removed and the solution was allowed to stir for 2 hours. Water was added, the layers separated, and the aqueous layer was extracted with EtOAc. The combined organic layers were washed with brine, dried (MgSO₄), filtered, and chromatographed on silica gel, eluting with acetone. The appropriate fractions were combined and evaporated to afford a white solid. The solid was recrystallized from hexane to afford racemic α-(2,3-dimethoxyphenyl)-1-[2-(4-fluorophenyl)ethyl]-4-piperidinemethanol as shiny white needles, m.p. 126-127° C. Anal. Calcd. for C₂₂H₂₈FNO₃: C, 70.75; H, 7.56; N, 3.75. Found: C, 70.87; H, 7.65; N, 3.68.

### EXAMPLE 2

Example 2, Steps A-F, demonstrate an alternative manner of preparing (±)-α-(2,3-dimethoxyphenyl)-1-[2-(4-fluorophenyl)ethyl]-4-piperidinemethanol, structure 1.

### A) 1-(1,1-Dimethylethyl)-1,4-piperidinedicarboxylic acid

To isonipecotic acid (107.5 g, 832 mmol) stirred in 1N NaOH (40 g NaOH in 900 mL H₂O) and tert-butanol (1800 mL) was added di-tert-butyl dicarbonate (200 g, 916 mmol) in portions. After stirring overnight, the solution was concentrated and the resulting water layer was extracted 3× with ether. The combined organic layers were washed with water, brine, dried (MgSO₄), filtered, and evaporated to a white solid, which was recrystallized from EtOAc/hexane (300 mL/200 mL) to afford 1-(1,1-dimethylethyl)-1,4-piperidinedicarboxylic acid as white needles, m.p. 147-149° C.

### B) 4-(N-methoxy-N-methylcarboxamido)-1-piperidinecarboxylic acid 1,1-dimethylethyl ester

To a stirred solution of 1-(1,1-dimethylethy)-1,4-piperidinedicarboxylic acid (50.0 g, 218 mmol) in anhydrous CH₂Cl₂ (500 mL) under N₂ in a 2 L flask was added 1,1′-carbonyldiimidazole (38.9 g, 240 mmol) portionwise. After stirring for 1 hour, N,O-dimethylhydroxylamine hydrochloride (23.4 g, 240 mmol) was added in one portion. After stirring overnight, the solution was washed twice with 1N HCl, twice with saturated NaHCO₃, once with brine, dried (MgSO₄), filtered, and evaporated to an oil. Distillation afforded 4-(N-methoxy-N-methylcarboxamido)-1-piperidinecarboxylic acid 1,1-dimethylethyl ester as a clear oil, b.p. 120-140° C., 0.8 mm.

### C) 4-(2,3-Dimethoxybenzoyl)-1-piperidinecarboxylic acid 1,1-dimethylethyl ester

n-Butyl lithium (14.5 mL of a 2.5 M solution in hexane, 36.3 mmol) was added via syringe to a stirred solution of veratrole (5.00 g, 36.2 mmol) in THF (50 mL, anhydrous) under argon at 0° C. The ice bath was removed and the mixture was allowed to stir for 90 minutes. The mixture was cooled to −78° C. and treated with 4-(N-methoxy-N-methylcarboxamido)-1-piperidinecarboxylic acid 1,1-dimethylethyl ester (9.20 g, 33.8 mmol) in THF (50 mL, anhydrous) via syringe. The cooling dry ice-acetone bath was removed and the mixture was allowed to come to room temperature. After stirring for 3 hours, saturated aqueous NH₄Cl was added and the mixture was allowed to stir overnight. The layers were separated and the aqueous layer was extracted with ether. The combined organic layers were washed with brine, dried (MgSO₄), filtered, and evaporated to afford an amber oil. The oil was chromatographed on silica gel, eluting with 20% EtOAc in hexane. The appropriate fractions were combined and evaporated to an amber oil. The oil was distilled to afford 4-(2,3-dimethoxybenzoyl)-1-piperidinecarboxylic acid 1,1-dimethylethyl ester as a colorless oil. (b.p. 225-250° C., 0.05 mm). Anal. Calcd. for C₁₉H₂₇NO₅: C, 65.31; H, 7.79; N, 4.01. Found: C, 65.04; H, 7.92; N, 4.11.

### D) 4-(2,3-Dimethoxyphenyl)-4-piperidinylmethanone

4-(2,3-Dimethoxybenzoyl)-1-piperidinecarboxylic acid 1,1-dimethylethyl ester (7.75 g, 22.2 mmol) was dissolved in trifluoroacetic acid (50 mL, 650 mmol) and stirred for 45 minutes. The entire solution was poured into ether (900 mL) and allowed to stand overnight. Filtration yielded 4-(2,3-dimethoxyphenyl)-4-piperidinylmethanone trifluoroacetate as fine white needles, m.p. 123° C. Anal. Calcd. for C₁₄H₁₉NO₃.CF₃CO₂H: C, 52.89; H, 5.55; N, 3.86. Found: C, 52.77; H, 5.62; N, 3.82.

The resulting 4-(2,3-dimethoxyphenyl)-4-piperidinylmethanone trifluoroacetate was dissolved in water, treated with NaOH (10% aqueous) until basic, and extracted three times with dichloromethane. The combined organic layers were washed with brine, dried (MgSO₄), filtered and evaporated to afford 4-(2,3-dimethoxyphenyl)-4-piperidinylmethanone as an oil.

### E) (2,3-Dimethoxyphenyl)[1-[2-(4-fluorophenyl)ethyl]-4-piperidinyl]methanone monohydrochloride

A solution of 4-(2,3-dimethoxyphenyl)-4-piperidinylmethanone (8.00 g, 32.1 mmol) and 2-(4-fluorophenyl)ethyl bromide (6.52 g, 32.1 mmol) was prepared in DMF (90 mL) treated with K₂CO₃ (7.0 g, 50.7 mmol), then stirred and heated at 80° C. under argon overnight. The cooled solution was poured into a partition of 2/1 EtOAc/toluene and water. The layers were separated and the aqueous layer was extracted with 2/1 EtOAc/toluene. The combined organic layers were washed 2× with water, 1× with brine, dried (MgSO₄), filtered, and evaporated to afford 11.0 g of an oil. The oil was chromatographed on silica gel, eluting with EtOAc. The appropriate fractions were combined, concentrated, dissolved in ethyl acetate and treated with HCl/ethyl acetate. (2,3-dimethoxyphenyl)[1-[2-(4-fluorophenyl)ethyl]-4-piperidinyl]-methanone monohydrochloride was obtained as a precipitate, m.p. 225-227° C. (decomp). Anal. Calcd. for C₂₂H₂₆FNO₃.HCl: C, 64.78; H, 6.67; N, 3.43. Found: C, 64.44; H, 6.73; N, 3.41.

### F) (±)-α-(2,3-Dimethoxyphenyl)-1-[2-(4-fluorophenyl)ethyl]-4-piperidinemethanol

To a stirred solution of (2,3-dimethoxyphenyl)[1-[2-(4-fluorophenyl)ethyl]-4-piperidinyl]-methanone (6.0 g, 16.2 mmol) in MeOH (100 mL) at 0° C. was added NaBH₄ (1240 mg, 32.8 mmol) in two portions, over an one hour period. After stirring overnight, the solution was concentrated to a solid. The solid was partitioned between water and ether. The layers were separated and the aqueous layer was extracted with ether. The combined organic layers were washed with brine, dried (MgSO₄), filtered, and evaporated to a solid. The solid was chromatographed on silica gel, eluting with acetone. The appropriate fractions were combined and evaporated to afford a white solid. The solid was recrystallized from cyclohexane to afford (O)-α-(2,3-dimethoxyphenyl)-1-[2-(4-fluorophenyl)-ethyl]-4-piperidinemethanol as white needles, m.p. 126-127° C. Anal. Calcd. for C₂₂H₂₈FNO₃: C, 70.75; H, 7.56; N, 3.75. Found: C, 70.86; H, 7.72; N, 3.93.

### EXAMPLE 3

This example demonstrates the preparation of the compound of Formula-I.

### Preparation of (+)-α-(2,3-Dimethoxyphenyl)-1-[2-(4-fluorophenyl)ethyl]-4-piperidinemethanol

### A) Preparation of diastereomers

A solution of 3.90 g (10.4 mmol) of (O)-α-(2,3-dimethoxyphenyl)-1-[2-(4-fluorophenyl)ethyl]-4-piperidinemethanol, 1.74 g (10.4 mmol) of S-(+)-α-methoxyphenylacetic acid, 2.15 g (10.4 mmol) of 1,3-dicyclohexylcarbodiimide and 0.1 g of 4-dimethylaminopyridine in chloroform (75 mL) was refluxed for 17 hours, allowing to cool to room temperature and filtered. The filtrate was concentrated and chromatographed on a silica gel column eluting with ethyl acetate/hexane (1:1) to afford two diastereomers, Rf=0.1 and 0.2 (TLC EtOAc/hexane, 1:1). Intermediate fractions were re-chromatographed to give additional material. Those fractions with Rf=0.2 were combined to give a single diastereomeric ester, (+,+)-(2,3-dimethoxyphenyl)[1-[2-(4-fluorophenyl)ethyl]-4-piperidinyl]methyl-α-methoxybenzene-acetate.

### B) Preparation of (+)-α-(2,3-dimethoxyphenyl)-1-[2-(4-fluorophenyl)ethyl]-4-piperidinemethanol

To a stirred solution of 0.97 g (1.9 mmol) of the above mentioned diastereomeric ester, Rf=0.2, in 25 mL of methanol was added 0.5 g (3.6 mmol) of potassium carbonate and 5.0 mL of water. After stirring 17 hours at room temperature the reaction mixture was diluted with water and extracted twice with methylene chloride. The combined extracts were washed with water, brine and dried over MgSO₄. After filtering, the filtrate was concentrated to an oil and crystallized from 40 mL of cyclohexane/hexane (1:1) to give (+)-α-(2,3-dimethoxyphenyl)-1-[2-(4-fluorophenyl)ethyl]-4-piperidinemethanol, m.p. 112-113° C., [α]_{D}²⁰=+13.9°.

### EXAMPLE 4

### Study of the Effects of a Combination of an Antagonist of the GABA Receptors and an Inhibitor of the 5HT2A Receptors in Improving the Quality of Sleep

For this study, four groups of male Sprague-Dawley rats are used, each group comprises 5 to 9 rats.

Group A receives 0.3 mg/kg i.p. Compound A (intraperitoneally)
Group B receives 3 mg/kg p.o. zolpidem (orally, hemitartarate)
Group C receives the combination—0.3 mg/kg i.p. Compound A and 3 mg/kg p.o. zolpidem hemitartarate, the two compounds are administered in 5-minute intervals orally or intraperitoneally, as noted.
Finally, group D receives 10 mg/kg p.o. zolpidem (orally, hemitartarate). The data are recorded on day 0 (reference date) when animals receive only a carrier (distilled water and methylcellulose) and on day 1 when animals receive the active principle. The data are recorded for 6 hours each day, active principles are administered 15 minutes after the beginning of the record.

The synergistic effects of the combination is measured by the decrease in the waking-up time (total waking-up time during the 6 hours of recordation), increase in the non-rapid eye movement (NREM) duration (total duration of NREM sleep during the 6 hours of recordation), and general decrease in the number of NREM sleep periods. Thus the combination of the invention enhances sleep quality in a patient.

### EXAMPLE 5

### Preparation of a Capsule Containing Compound A and Zolpidem

A capsule is prepared containing, in the form of a small size tablet, 1.18 mg Compound A as sleep aid and 6.22 mg zolpidem hemitartarate as a short-acting hypnotic agent. The tablet contains the ingredients as listed in Table I below.

TABLE I

| Ingredient | Percent by Weight |
|---|---|
| Micronized Compound A | 2.36 |
| Monohydrated lactose¹ | 87.14 |
| Gelatinized Starch² | 8 |
| Sodium croscarmellose³ | 2 |
| Magnesium stearate | 0.5 |

¹Pharmatose DMV
²Starch 1500
³Ac-di-sol (FMC)

First the mixture of Compound A, monohydrated lactose, Gelatinized Starch, sodium croscaramellose and magnesium stearate is prepared. The mixture is then placed in biconic mixer for thirty minutes. The homogenous mixture is then compressed, by using a normal rotary compressed machine, in the form of 50 mg tablet.

The zolpidem hemitartarate tablet is prepared using the ingredients shown in Table II below.

TABLE II

| Ingredient | Percent by Weight |
|---|---|
| Zolpidem hemitartarate | 10.37 |
| Lactose | 83.73 |
| Microcrystalline cellulose⁴ | 10.0 |
| Hydroxypropylmethylcellulose 606⁵ | 2.1 |
| Sodium carboxymethylcellulose | 3.2 |
| Magnesium stearate | 0.6 |

⁴Avicel (FMC)
⁵Pharmacoat 606 (Shin-Etsu)

The Zolpidem hemitartarate, lactose, microcrystalline cellulose, hydroxypropylmethylcellulose and sodium carboxymethylcellulose are mixed together, and then are granulated with water. The granulate is then dried and calibrated. The granulate is then mixed with the magnesium stearate and compressed in a mass of 60 mg per tablet, by using rotary compressed machine.

Then, tablets with a dose of 1 mg of Compound A and 6.42 mg of zolpidem hemitartarate are introduced in a hard gelatin capsule. The capsules dissolution profiles can be measured by using a II device of the US Pharmacopoeia, with two dissolution medium:
900 ml of hydrochloric acid 0.01 M and
900 ml of potassium phosphate buffer 0.05 M at pH 6,8, maintained at 37+/−0.5° C., with stirring (50 t.p. min.)

### EXAMPLE 6

### Preparation of a Capsule Containing an Immediate-Release Compound a Tablet and a Delayed Release Zolpidem Tablet

The immediate release Compound A tablets are prepared according to the process described in Example 5 above.

The delayed release zolpidem hemitartarate tablet is prepared according to the method described in Example 5 above in order to obtain a tablet having the composition indicated in Table III below.

TABLE III

| Ingredients | Percent by Weight |
|---|---|
| Zolpidem hemitartarate | 12.4 |
| Monohydrated lactose⁶ | 33.4 |
| Hydroxypropylmethylcellulose 4000 mPa · s⁷ | 25.0 |
| Microcrystalline cellulose⁸ | 20.0 |
| Hydrogen potassium tartrate | 8.0 |
| Magnesium stearate | 1.0 |
| Colloidal anhydrous silica | 0.2 |
| Purified water | q.s. |

⁶Pharmatose (DMV)
⁷Metolose 90SH4000 (Shin-Etsu)
⁸Avicel PH 102 (FMC)

The same humid granulation and compression methods are used, such as those described for the zolpidem hemitartarate in Example 5 above. Capsules are prepared containing one or more of the 50 mg delayed release tablets containing 5 mg of zolpidem base (corresponding to 6.22 mg of zolpidem hemitartarate) and one more of the 50 mg immediate-release tablets containing 1 mg of Compound A.

The in vitro dissolution profiles of the capsules prepared like this can be established by using the method described in Example 5 above

### EXAMPLE 7

### Preparation of a Capsule Comprising a Mixture of Immediate-Release Compound a Pellets and of Immediate-Release Zolpidem Pellets

A suspension of 50 g of Compound A and of 100 g of povidone (Pladone K29/32, BASF) in 670 g of ethanol is prepared. 750 g of that suspension are then pulverized on 1060 g of microgranules of 16-18 mesh size, by using a fluidized bed dryer. Then, a suspension of 62.2 g of zolpidem tartrate (corresponding to 50 g of zolpidem base) and of 100 g of povidone (Pladone K29/32, BASF) in 670 g of ethanol is prepared. 750 g of that suspension are then pulverized on 1060 g of microgranules of 16-18 mesh size, by using a fluidized bed dryer. A mixture of the two pellets is prepared, with a ratio of 1 part in weight of Compound A for 5 part of zolpidem tartrate. This mixture is put in a hard gelatin capsule having a total quantity of 1 mg of Compound A and 5 mg of zolpidem in the base form (corresponding to 6.22 mg of zolpidem tartrate). The quantity of each of the pellets can be modified in order to adjust the dose.

The in vitro dissolution profiles of the capsules prepared like this can be established by using the method described in Example 5 above.

### EXAMPLE 8

### Preparation of a Capsule Comprising a Mixture of Immediate-Release Compound a Pellets and of Delayed Release Zolpidem Pellets

The immediate-release Compound A pellets are prepared according to the method described in Example 7 above. Similarly, Zolpidem hemitartarate pellets are prepared such as described above in Example 5.

A solution comprising 25 g of methacrylate copolymer (Eudragit TM RL 100, Rohm Pharma), 143 g of methacrylate copolymer (Eudragit TM RS 100, Rohm Pharma) and 18.7 g of ethyl citrate (Eudrafex TM, Rohm Pharma) is prepared in a 1180 g isopropanol/acetone 60:40 (wt/wt) mixture. The zolpidem hemitartarate pellets are coated with this mixture of polymers, by pulverization in a fluidized bed dryer, the final quantity of coating represents 20% by weight of the non coated pellet mass. After saturation of pellets at 35° C. for 24 hours, a mixture of coated zolpidem hemitartarate pellets and Compound A pellets is prepared, in the proportion of 1:2 (Compound A/zolpidem), and this mixture is put in gelatin capsules in order to give a quantity per capsule corresponding to 5 mg of Compound A and 10 mg of zolpidem base.

The in vitro dissolution profiles of the capsules prepared like this can be established by using the method described in Example 5 above.

### EXAMPLE 9

### Preparation of a Tablet Comprising Immediate-Release Compound a Pellets and Immediate-Release Zolpidem Pellets

Compound A and zolpidem hemitartarate pellets are prepared according to the method described in Example 7 above.

A mixture by weight of the two pellets is prepared in a ratio of 1 part of Compound A for 2 parts of zolpidem hemitartarate, and 0.1% of magnesium stearate is added. The mixture is then placed in a biconical mixer for 30 minutes.

The homogenous mixture is then compressed by using a conventional rotary compression machine, in order to give a tablet having 5 mg of Compound A and 12.44 mg of zolpidem hemitartarate (corresponding to 10 g of zolpidem in the base form). The in vitro dissolution profiles of the capsules prepared like this can be established by using the method described in Example 5 above.

### EXAMPLE 10

### Preparation of a Tablet Comprising Immediate-Release Compound a Pellets and Delayed-Release Zolpidem Pellets

The immediate-release Compound A pellets are prepared according to the method described in Example 7 and the delayed release zolpidem hemitartarate pellets according to the method described in Example 8.

A mixture of the two pellets is prepared in a ratio of 2 parts of Compound A and 6 parts of zolpidem hemitartarate, and 0.2% of magnesium stearyl fumarate are added. The mixture is then transferred into a biconical mixer for 30 minutes. The homogenous mixture is then compressed by using a conventional rotary compression machine, in order to give a total quantity of 4 mg of Compound A and 14.93 mg of zolpidem hemitartarate (corresponding to 12 g of zolpidem base). The in vitro dissolution profiles of the capsules prepared like this can be established by using the method described in Example 5 above.

### EXAMPLE 11

### Preparation of a Delayed Release Enteric Tablet Comprising Immediate-Release Compound a Pellets and Immediate-Release Zolpidem Pellets

Tablets are prepared comprising both Compound A and zolpidem hemitartarate according to the process described in Example 9. Tablets are then coated according to the process known by the person skilled in the art and described hereafter.

A solution of 46 g of methacrylate copolymer (Eudragit TM RL100, Rohm Pharma), 295 g of methacrylate copolymer (Eudragit TM RS100, Rohm Pharma) and 40 g of ethyl citrate (Eudrafex TM, Rohm Pharma) in 2280 g of a mixture isopropanol/acetone 65:35 (wt/wt) is prepared.

The tablets comprising 3.2 mg of Compound A and 12.44 mg of zolpidem hemitartarate are coated with polymeric mixture, by pulverization in a coating pan, the final quantity of the coating represents 5 to 10% in weight of the pellet mass without coating.

### EXAMPLE 12

### Preparation of a Bilayer Tablet Comprising an Immediate-Release Compound a Layer and an Immediate-Release Zolpidem Layer

Granulates A are prepared by dry mixture and granulates B by wet mixture according to Example 5 using the compositions as listed in Table IV below.

TABLE IV

| Ingredients | Percent by Weight |
|---|---|
| Granulates A | |
| Compound A | 2.95 |
| Dry monohydrated lactose⁹ | 82.71 |
| Pregelatinized Starch¹⁰ | 8.00 |
| Croscarmellose¹¹ | 2.00 |
| Sodium carboxymethylcellulose¹² | 3.80 |
| Magnesium stearate¹³ | 0.54 |
| Granulates B | |
| Zolpidem hemitartarate | 6.22 |
| Monohydrated lactose⁹ | 73.88 |
| Microcrystalline cellulose¹⁴ | 14.0 |
| Hydroxypropylmethylcellulose 606¹⁵ | 2.1 |
| Sodium carboxymethylcellulose¹² | 3.2 |
| Magnesium stearate¹³ | 0.6 |

⁹Pharmatose (DMV)
¹⁰Starch 1500 (Colorcon)
¹¹Ac-di-sol (FMC)
¹²Blanose (Aqualon)
¹³Brentag AG
¹⁴Avicel PH 102 (FMC)
¹⁵Pharmacoat 606 (Shin-Etsu)

The mixtures are then compressed into a bilayer tablet by using an alternative compression machine, the first immediate-release layer with a 200 mg mass of granulate A comprising 5 mg of Compound A and the second immediate-release layer with a 200 mg mass of granulate B comprising 12.44 mg of zolpidem hemitartarate (corresponding to 10 mg of zolpidem base).

The in vitro dissolution profiles of the capsules prepared like this can be established by using the method described in Example 5 above.

### EXAMPLE 13

### Preparation of a Bilayer Tablet Comprising an Immediate-Release Compound a Layer and a Delayed Release Zolpidem Layer

Granulates C are prepared by dry mixture and granulates D by wet mixture according to Example 5 using the compositions as listed in Table V below.

TABLE V

| Ingredients | Percent by Weight |
|---|---|
| Granulates C | |
| Compound A | 2.95 |
| Dry monohydrated lactose¹⁶ | 84.00 |
| Pregelatinized Starch¹⁷ | 7.70 |
| Croscarmellose¹⁸ | 2.00 |
| Sodium carboxymethylcellulose¹⁹ | 3.4 |
| Magnesium stearate²⁰ | 0.54 |
| Granulates D | |
| Zolpidem hemitartarate | 7.75 |
| Lactose 150 mesh¹⁶ | 37.85 |
| Microcrystalline cellulose²¹ | 20.0 |
| Tartaric acid (23) | 8.4 |
| Hydroxypropylmethylcellulose²² | 25.0 |
| Magnesium stearate²³ | 1.0 |

¹⁶Pharmatose (DMV)
¹⁷Starch 1500 (Colorcon)
¹⁸Ac-di-sol (FMC)
¹⁹Blanose (Aqualon)
²⁰Brentag AG
²¹Avicel PH 102 (FMC)
²²Metolose 90SH4000 (Shin-Etsu)
²³Brentag AG

The mixtures are then compressed into a bilayer tablet by using an alternative compression machine, the first immediate-release layer with a 150 mg mass of granulate C comprising 3.75 mg of Compound A and the second delayed release layer with a 200 mg mass of granulate D comprising 15.50 mg of zolpidem hemitartarate (corresponding to 12.45 mg of zolpidem base).

The in vitro dissolution profiles of the capsules prepared like this can be established by using the method described in Example 5 above.

### EXAMPLE 14

### Preparation of a Three Layers Tablet Comprising One Immediate-Release Compound A, One Inactive Layer and a Third Delayed Release Zolpidem Layer

Granulates E and F are prepared by dry mixture and granulates G by wet mixture according to Example 5 and using the compositions listed in table VI below.

TABLE VI

| Ingredients | Percent by Weight |
|---|---|
| Granulates E (immediate release) | |
| Compound A | 2.36 |
| Dry monohydrated lactose²⁴ | 87.14 |
| Pregelatinized Starch²⁵ | 8.0 |
| Croscarmellose²⁶ | 2.0 |
| Sodium carboxymethylcellulose²⁷ | 3.8 |
| Magnesium stearate²⁸ | 0.54 |
| Granulates F (inactive) | |
| Dry monohydrated lactose²⁴ | 60.0 |
| Microcrystalline cellulose²⁹ | 24.0 |
| Tartaric acid³⁰ | 10.0 |
| Hydroxyethylcellulose | 5.0 |
| Magnesium stearate²⁸ | 1.0 |
| Granulates G (delayed release) | |
| Zolpidem hemitartarate | 5.0 |
| Lactose 200 mesh²⁴ | 67.7 |
| Microcrystalline cellulose²⁹ | 20.0 |
| Hydroxypropylmethylcellulose 606³¹ | 2.5 |
| Sodium carboxymethylcellulose²⁷ | 3.8 |
| Magnesium stearate²⁸ | 1.0 |

²⁴Pharmatose (DMV)
²⁵Starch 1500 (Colorcon)
²⁶Ac-di-sol (FMC)
²⁷Blanose (Aqualon)
²⁸Brentag AG
²⁹Avicel PH 102 (FMC)
³⁰Brentag AG
³¹Pharmacoat (Shin-Etsu)

The mixtures are compressed, according to Example 12, into a three layers tablet, a 125 mg mass external layer of granulate E comprising 2.5 mg of Compound A, a 125 mg intermediary layer of granulate F and a third 300 mg mass external layer of granulate G comprising 15 mg of zolpidem hemitartarate (corresponding to 12.06 mg of zolpidem base).

### EXAMPLE 15

### Preparation of a Dry Coated Tablet Comprising an Internal Core of Zolpidem and an External Coating of Compound A

Granulates are prepared according to Example 5, and based on the compositions listed in table VII below.

TABLE VII

| Ingredients | Percent by Weight |
|---|---|
| Internal core (delayed release) | |
| Zolpidem hemitartarate | 15.55 |
| Monohydrated lactose 200 mesh³² | 36.05 |
| Microcrystalline cellulose³³ | 18.0 |
| Hydroxypropylmethylcellulose³⁴ | 21.0 |
| Tartaric acid³⁵ | 8.4 |
| Magnesium stearate³⁵ | 1.0 |
| External coating (immediate release) | |
| Compound A | 1.96 |
| Monohydrated lactose 150 mesh³² | 52.00 |
| Microcrystalline cellulose³³ | 39.84 |
| Hydroxypropylmethylcellulose 606³⁴ | 2.2 |
| Sodium carboxymethylcellulose³⁶ | 3.0 |
| Magnesium stearate³⁵ | 1.0 |

³²Pharmatose (DMV)
³³Avicel PH 102 (FMC)
³⁴Metolose 90SH4000 (Shin-Etsu)
³⁵Brentag AG
³⁶Blanose (Aqualon)

The granulate forming the internal core is compressed, by using an alternative compression machine, in little tablets, before performing the dry coating operation with the second layer. This operation produces 80 mg delayed release tablets, containing 12.44 mg of zolpidem hemitartarate (corresponding to 10 mg of zolpidem base).

The granulate forming the external coating layer is compressed, by using an alternative compression machine that allows the little internal core tablets. The external layer has a mass of 301 mg and contains 5 mg of Compound A.

According to another of its aspects, the object of the invention is to use at least one long-acting hypnotic agent and/or a sleep aid in combination with at least one short-acting hypnotic agent, for the preparation of a medication aimed to prevent and/or to treat the sleep disorders.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A combination comprising at least one short acting hypnotic agent and R-(+)-α-(2,3-dimethoxyphenyl)-1-[2-(4-fluorophenyl)ethyl]-4-piperidinemethanol (Compound A) or its prodrug having the Formula II: 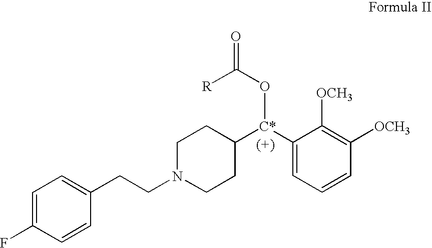
wherein R is C₁-C₂₀ alkyl or a pharmaceutically acceptable salt thereof.

2. The combination as set forth in claim 1, wherein the short-acting hypnotic agent is present in a galenic form adapted for an immediate or delayed release, and Compound A is present in a galenic form adapted for an immediate release.

3. The combination as set forth in claim 1, wherein the short-acting hypnotic agent is a modulator of GABA-A receptors, benzodiazepine, a melatonin derivative, or an agonist of melatonin receptors.

4. The combination as set forth in claim 1, wherein the short-acting hypnotic agent is selected from the group consisting of zolpidem, zopiclone, eszopiclone, zaleplon, melatonin, ramelteon, triazolam, etizolam, brotizolam and indiplon or a derivative or a mixture in any combination thereof.

5. The combination as set forth in claim 1, wherein the short acting hypnotic agent is zolpidem or a pharmaceutically acceptable salt thereof which is in combination with Compound A.

6. A pharmaceutical composition comprising at least one short acting hypnotic agent and R-(+)-α-(2,3-dimethoxyphenyl)-1-[2-(4-fluorophenyl)ethyl]-4-piperidinemethanol (Compound A) or its prodrug having the Formula II: 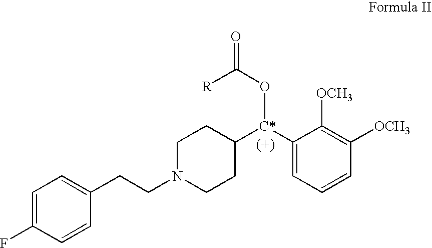
wherein R is C₁-C₂₀ alkyl or a pharmaceutically acceptable salt thereof in combination with one or more pharmaceutically acceptable diluent, excipient or a carrier.

7. The composition as set forth in claim 6, wherein the short-acting hypnotic agent is present in a galenic form adapted for an immediate or delayed release, and Compound A is present in a galenic form adapted for an immediate release.

8. The composition as set forth in claim 6, wherein the short-acting hypnotic agent is a modulator of GABA-A receptors, benzodiazepine, a melatonin derivative, or an agonist of melatonin receptors.

9. The composition as set forth in claim 6, wherein the short-acting hypnotic agent is selected from the group consisting of zolpidem, zopiclone, eszopiclone, zaleplon, melatonin, ramelteon, triazolam, etizolam, brotizolam and indiplon or a derivative or a mixture in any combination thereof.

10. The composition as set forth in claim 6, wherein the short-acting hypnotic agent is zolpidem or a pharmaceutically acceptable salt thereof which is in combination with Compound A.

11. The composition as set forth in claim 6, wherein the short-acting hypnotic agent and Compound A are released immediately.

12. The composition as set forth in claim 6, wherein the short-acting hypnotic agent has a delayed release and Compound A is released immediately.

13. The composition as set forth in claim 6, which consists in a capsule comprising one or more immediate-release tablets containing the short-acting hypnotic agent and one or more immediate-release tablets containing Compound A.

14. The composition as set forth in claim 6, which consists in a capsule comprising one or more delayed-release tablets containing the short-acting hypnotic agent and one or more immediate-release tablets containing Compound A.

15. The composition as set forth in claim 6, which consists in a capsule comprising a mixture of immediate-release pellets with the short-acting hypnotic agent and immediate-release pellets with Compound A.

16. The composition as set forth in claim 6, which consists in a capsule comprising a mixture of delayed-release pellets with the short-acting hypnotic agent and immediate-release pellets with Compound A.

17. The composition as set forth in claim 6, which consists in a tablet containing immediate-release pellets of the short-acting hypnotic agent and Compound A.

18. The composition as set forth in claim 6, which consists in a tablet containing delayed-release pellets of the short-acting hypnotic agent and immediate-release pellets of Compound A.

19. The composition as set forth in claim 6, which consists in a delayed-release enteric coated tablet comprising immediate-release pellets of Compound A and immediate-release pellets of the short-acting hypnotic agent.

20. The composition as set forth in claim 6, which consists in a multilayer tablet comprising:
(a) one or more immediate-release layers, each one containing a dose of Compound A and optionally a dose of short-acting hypnotic agent,
(b) one or more delayed-release layers, each one containing a dose of short-acting hypnotic agent and optionally Compound A, and
(c) an inactive layer.

21. The composition as set forth in claim 6, which consists in a dry coated tablet, comprising a delayed-release inner core containing Compound A and in that the immediate-release coating layer contains the short acting hypnotic agent.

22. A method of treating a sleep disorder in a patient comprising administering to said patient a therapeutically effective amount of a combination comprising at least one short acting hypnotic agent and R-(+)-α-(2,3-dimethoxyphenyl)-1-[2-(4-fluorophenyl)ethyl]-4-piperidinemethanol (Compound A) or its prodrug having the Formula II: 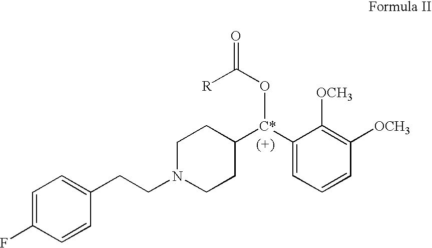
wherein R is C₁-C₂₀ alkyl or a pharmaceutically acceptable salt thereof.

23. The method as set forth in claim 22, wherein the sleep disorder is insomnia.

24. The method as set forth in claim 22, wherein the sleep disorder is primary insomnia.

25. The method as set forth in claim 22, wherein the sleep disorder is sleep maintenance insomnia.

26. The method of claim 22, wherein the sleep disorder is insomnia related to another mental disorder.

27. The method as set forth in claim 22, wherein the sleep disorder is substance induced insomnia.

28. The method as set forth in claim 22, wherein the sleep disorder is obstructive sleep apnea insomnia.

29. The method as set forth in claim 22, wherein the short-acting hypnotic agent is selected from the group consisting of zolpidem, zopiclone, eszopiclone, zaleplon, melatonin, ramelteon, triazolam, etizolam, brotizolam and indiplon or a derivative or a mixture in any combination thereof.

30. The method as set forth in claim 22, wherein the combination is Compound A and zolpidem or a pharmaceutically acceptable salt thereof.
